(19) United States
(12) Patent Application Publication
(10) Pub. No.: US 2007/0118063 A1
(43) Pub. Date: May 24, 2007

(54) Method and Apparatus for Leukoreduction of Red Blood Cells

(75) Inventor: Bruce GIBBS, Arvada, CO (US)

Correspondence Address: GAMBRO, INC;PATENT DEPARTMENT, 10810 W COLLINS AVE, LAKEWOOD, CO, 80215, US

(73) Assignee: GAMBRO, INC, Lakewood, CO (US)

(21) Appl. No.: 11/538,993

(22) Filed: Oct. 5, 2006

Related U.S. Application Data
(60) Provisional application No. 60/596,591, filed on Oct. 5, 2005.

(51) Int. Cl. A61M 37/00

(52) U.S. Cl. 604/6.02

(57) ABSTRACT

This invention relates to a method for the continuous separation of red blood cells from whole blood which includes the steps of removing the whole blood from a donor, continuously separating the whole blood into blood component layers within a separation vessel including at least a plasma layer, a buffy coat layer and a red blood cell layer, removing the plasma layer from the separation vessel, removing the buffy coat layer from the separation vessel, and removing red blood cells from the red blood cell layer which partially overlaps with the buffy coat layer to create a mononuclear cell (MNC)-reduced red blood cell layer in the separation vessel.

### PRIORITY CLAIM

This invention claims the benefit of U.S. provisional application No. 60/596,591 filed Oct. 5, 2005.

### FIELD OF INVENTION

The present invention relates generally to the field of extracorporeal blood processing methods which are particularly useful in blood component collection, and more particularly, the present invention relates to methods for the leukoreduction of red blood cells collected with an apheresis system.

### BACKGROUND OF THE INVENTION

One well-known type of extracorporeal blood processing involves an apheresis system and/or procedure in which blood is removed from a donor or a patient (hereafter cumulatively referred to as a donor), directed to a blood component separation device (e.g., centrifuge), and separated into various blood component types (e.g., red blood cells, white blood cells, platelets, plasma) for collection or therapeutic purposes. One or more or all of these blood component types may be collected, and/or treated for therapeutic purposes before storage or return to a patient, while the remainder may simply be returned to the donor or patient.

A number of factors may affect the commercial viability of an apheresis system. One factor relates to the time and/or expertise required of an individual to prepare and operate the apheresis system. For instance, reducing the time required by the operator to complete an entire collection procedure, as well as reducing the complexity of these actions, can increase productivity and/or lower the potential for operator error. Moreover, reducing the dependency of the system on the operator may further lead to reductions in the credentials desired/required for the operators of these systems.

Donor-related factors may also impact the commercial viability of an apheresis system and include, for example, donor convenience and donor comfort. For instance, donors/patients may have a limited amount of time which may be committed to a donation or therapeutic procedure. Consequently, once at the collection or treatment facility, the amount of time which is actually spent collecting and/or treating blood components is an important consideration. This also relates to donor comfort as the actual collection procedure may be somewhat discomforting because at least one and sometimes two access needles are disposed in the donor throughout the procedure.

Performance-related factors also affect the commercial viability of an apheresis system. Performance may be judged in terms of the collection efficiency of the apheresis system, which may impact or improve product quality and/or may in turn reduce the amount of processing time and thus decrease operator burden and increase donor convenience. The collection efficiency of a system may be gauged in a variety of ways, such as by the amount of a particular blood component type which is collected in relation to the quantity of this blood component type which passes through the apheresis system. Individual characteristics of the donor also contribute to the performance of apheresis systems, for example, some donors have greater percentages of certain blood cell types than other donors.

Performance may also be evaluated based upon the effect which the apheresis procedure has on the various blood component types. For instance, it is desirable to minimize the adverse effects on the blood component types as a result of the apheresis procedure (e.g., reduce platelet activation).

Another performance-related factor is the end quality of the collected blood component. For example, if red blood cells are the component to be collected, it is generally desirable that such red blood cells be leukoreduced by the removal of white blood cells or leukocytes. Contaminating white blood cells can present problems to the ultimate recipient of the collected blood component, by provoking immunogenic reactions and viral diseases.

Conventionally, filters have been used to remove leukocytes from collected blood products or components. For example, U.S. Pat. No. 5,954,971 discloses the use of a filter with an apheresis system for filtering a diluted blood component prior to collection. Other distinctive methods have also been used, and these have generally dictated special preliminary steps such as pre-chilling and/or overnight storage of collected components prior to filtration. Another distinct conventional filtration step is the venting or air handling/re-circulation or by-passing at the end of the filtration procedure which had been deemed important for substantial recovery of a remainder portion of the blood component to be processed through a red blood cell filter.

Leukocytes are made up of mononuclear cells and polymorphonuclear cells. Mononuclear cells consist of lymphocytes, monocytes and stem cells. Polymorphonuclear cells consist of granulocytes, eosinophils and basophils. As discussed above, a performance related factor, which may affect apheresis efficiency, is the amount of a particular cell component a donor has. For example, it has been observed that if a donor has a high percentage of lymphocytes as compared to other white blood cell subtypes, (or has a high lymphocyte load), leukofiltration is not as effective as in donors who do not have such high lymphocyte loads. There is often a high residual population of lymphocytes which are not removed via filtration and which contaminate the separated red blood cell component.

The present invention is directed towards removing mononuclear cells in an apheresis procedure before leukoreducing the separated blood components.

### SUMMARY OF THE INVENTION

The present invention relates to the extracorporeal separation and collection of red blood cells using an apheresis blood processing system. More particularly, this invention relates to a method for the continuous separation of red blood cells from whole blood wherein the portion of the red blood cells which are closest to the layer containing lymphocytes are collected within the blood processing vessel and are returned to the donor, along with the lymphocytes in the buffy coat, leaving the mononuclear cell reduced red blood cells within the separation vessel.

According to the present invention, before the ultimate collection of the red blood cells in the collection container, the MNC-reduced red blood cells are filtered through a filtration device. This filtration preferably occurs during the overall separation procedure, although it could be initiated soon after and as part of the commencement of the collection procedure. Nevertheless, the separation procedure may be a continuous or batch process, and in either case, the filtration occurs upon or soon after removal of the separated high hematocrit MNC-reduced red blood cells from the processing vessel, yet preferably concurrently with or soon after the overall separation process.

These and still further aspects of the present invention are more particularly described in the following description of the preferred embodiments presented in conjunction with the attached drawings which are described briefly below.

### BRIEF DESCRIPTION OF THE DRAWINGS

FIG. 1 is a schematic view of one embodiment of an apheresis system which can be used in or with the present invention.

FIG. 2 is a partial cross-sectional view of a portion of the separation vessel.

FIG. 3 illustrates a tubing and bag set including an extracorporeal tubing circuit, a cassette assembly, and a filter and collection bag assembly for use in or with the system of FIG. 1 pursuant to the present invention.

FIG. 4 illustrates a cassette assembly similar to that shown in the set of FIG. 3.

FIG. 5 illustrates a filter and collection bag assembly similar to that shown in the set of FIG. 3.

FIGS. 6A and 6B illustrate alternative filter and collection bag assemblies also usable in a tubing and bag set like that shown in FIG. 3.

FIG. 7 is a schematic view of an apheresis system according to the present invention.

FIG. 8 is a schematic view of an alternative apheresis system also according to the present invention.

FIG. 9 is a schematic view of an alternative apheresis system according to the present invention.

### DETAILED DESCRIPTION

The present invention will be described in relation to the accompanying drawings which assist in illustrating the pertinent features hereof. Generally, the primary aspects of the present invention relate to both procedural and structural improvements in or a sub-assembly for use with a blood apheresis system. However, certain of these improvements may be applicable to other extracorporeal blood processing applications whether any blood components are returned directly to the donor or otherwise; and such are within the scope of the present invention as well.

It should be noted that like elements are depicted by like numbers.

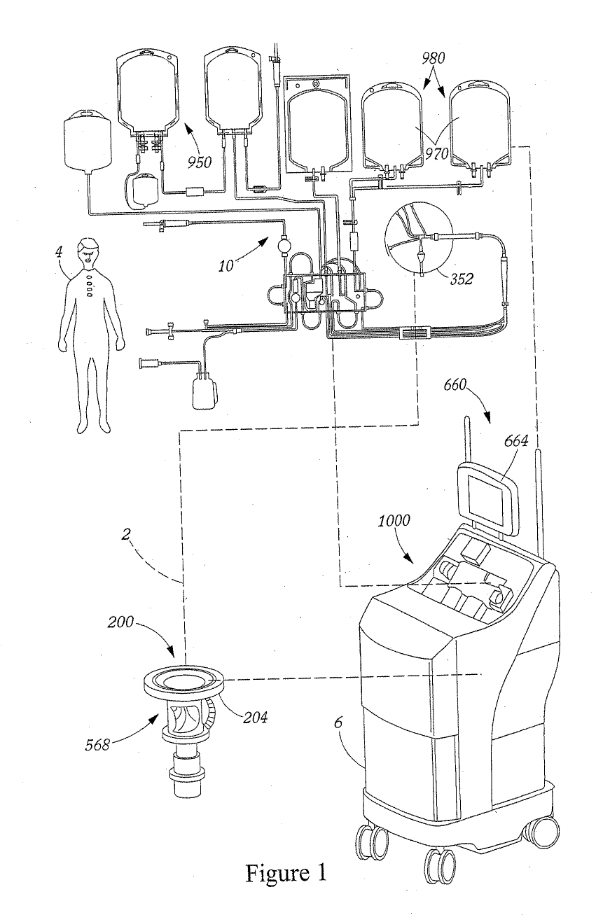

A preferred blood apheresis system 2 for use in and/or with the present invention is schematically illustrated in FIG. 1. System 2 provides for a continuous blood component separation process. Generally, whole blood is withdrawn from a donor 4 and is substantially continuously provided to a blood component separation device 6 where the blood is continuously separated into various component types according to density and at least one of these blood component types is preferably continuously collected from the device 6. One or more of the separated blood components may then either be provided for collection and subsequent use by another through transfusion or may be returned to the donor 4. Therapeutic treatment and near immediate return of certain separated blood components is a viable alternative use hereof as well. It is also understood that for therapeutic treatment the blood may be separated into components with filtration using the principles of the instant invention and as described below at a patient's bedside for return to such patient.

In the blood apheresis system 2, blood is withdrawn from the donor 4 and directed through a preconnected extracorporeal tubing circuit 10 and, in one embodiment, a blood processing vessel 352 which together define a closed, sterile and disposable system. The set 10 is preferably disposable and is adapted to be mounted on and/or in the blood component separation device 6. The separation device 6 preferably includes a pump/valve/sensor assembly 1000 for interfacing with the extracorporeal tubing circuit 10, and a channel assembly 200 for interfacing with the disposable blood processing vessel 352.

The channel assembly 200 may include a channel housing 204 which is rotatably interconnected with a rotatable centrifuge rotor assembly 568 which provides the centrifugal forces required to separate blood into its various blood component types by centrifugation. The blood processing vessel 352 may then be interfitted within the channel housing 204. When connected as described, blood can then be flowed substantially continuously from the donor 4, through the extracorporeal tubing circuit 10, and into the rotating blood processing vessel 352. The blood within the blood processing vessel 352 may then be continuously separated into various blood component types and at least one of these blood component types (platelets, plasma, lymphocytes or red blood cells) is preferably continually removed from the blood processing vessel 352. Blood components which are not being retained for collection or for therapeutic treatment are preferably also removed from the blood processing vessel 352 and returned to the donor 4 via the extracorporeal tubing circuit 10. Note, various alternative apheresis systems (not shown) may also make use of the present invention; including batch processing systems (non-continuous inflow of whole blood and/or non-continuous outflow of separated blood components) or smaller scale batch or continuous RBC/plasma separation systems, whether or even if no blood components may be returned to the donor.

Operation of the blood component separation device 6 is preferably controlled by one or more processors included therein, and may advantageously comprise a plurality of embedded computer processors to accommodate interface with ever-increasing PC user facilities (e.g., CD ROM, modem, audio, networking and other capabilities). Relatedly, in order to assist the operator of the apheresis system 2 with various aspects of its operation, the blood component separation device 6 preferably includes a graphical interface 660 with an interactive touch screen 664.

Further details concerning the operation of a preferred apheresis system, such as the Gambro Trima® System and the Trima Accel® System (available from Gambro BCT, Inc., Lakewood, Colo.) may be found in a plurality of publications, including, for example, WO99/11305 and U.S. Pat. No. 5,653,887; No. 5,676,644; No. 5,702,357; No. 5,720,716; No. 5,722,946; No. 5,738,644; No. 5,750,025; No. 5,795,317; No. 5,837,150; No. 5,919,154; No. 5,921,950; No. 5,941,842; No. 6,129,656; and No. 6,730,055 among numerous others. The disclosures hereof are incorporated herein as if fully set forth. A plurality of other known apheresis systems may also be useful herewith, as for example, the Baxter CS3000® and/or Amicus® and/or Autopheresis-C® and/or Alyx systems, and/or the Haemonetics MCS® or MCS®+ and/or the Fresenius COM.TEC™ or AS-104™ and/or the system described in Pat. No. 6,773,389.

Separation Vessel

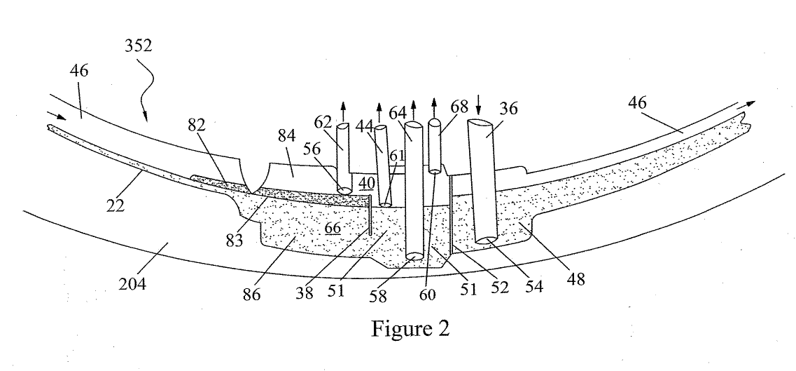

FIG. 2 schematically illustrates a portion of the separation vessel 352. FIG. 2 also illustrates an inflow tube 36 for conveying the whole blood to be separated into the separation vessel 352; first, second, and third collection lines 64, 62, 68 for removing separated substances from the separation vessel 352; and an interface control line 44 for adjusting the level of an interface between separated substances in the vessel 352. Preferably, the separation vessel 352 forms what is known as a single stage component separation area rather than forming a plurality of such stages. In other words, each of the components separated in the vessel 352 are collected and removed in only one area of the vessel 352. In addition, the separation vessel 352 includes a substantially constant radius except in the outlet portion 51 where the outer wall of the outlet portion is preferably positioned further away from the axis of rotation to allow for the ports 56, 58, 60, and 61 be positioned at different radial distances and to create a collection pool with greater depth for the high density red blood cells.

Although the ports 56, 58, and 60 and lines 62, 64, and 68 are referred to as being “collection” ports and lines the substances removed through these ports and lines can be either collected or reinfused back into a donor.

The separation vessel 352 has a generally annular flow path 46 and includes an inlet portion 48 and outlet portion 51. A wall 52 prevents substances from passing directly between the inlet and outlet portions 48 and 51 without first flowing around the generally annular flow path 46 (e.g., counterclockwise as illustrated by arrows in FIG. 2).

Although FIG. 2 shows the inlet portion 48 as having a wide radial cross-section, the outer wall of the inlet portion 48 can be spaced closer to the inner wall of the inlet portion 48 and/or tapered. An inlet port 54 of inflow tube 36 allows for flow of whole blood, into the inlet portion 48 of separation vessel 352. During a separation procedure, substances entering the inlet portion 48 follow the flow path 46 and stratify according to differences in density in response to rotation of the rotor 568. Preferably, the flow path 46 between the inlet and outlet portions 48 and 50 is curved and has a substantially constant radius. In addition, the flow path 46 is placed at the maximum distance from the axis of rotation. This shape ensures that components passing through the flow path 46 encounter a relatively constant gravitational field and a maximum possible gravitational field for the rotor 568.

The separated substances flow into the outlet portion 51 where they are removed via first 56, second 58, and third 60 collection ports respectively, of first 62, second 64, and third 68 collection lines. Separated substances may also be removed by an interface control port 61 of the interface control line 44. As shown in FIG. 2, the first 56, second 58, and third 60 ports are positioned at varying radial locations on the rotor 568 to remove substances having varying densities. The second collection port 58 is farther from the axis of rotation than the first and third ports 56 and 60 to remove the most dense substances separated in the separation vessel 352, such as red blood cells. The third port 60 is located closer to the axis of rotation than the first and second ports 56 and 58 to remove the least dense substances separated in the separation vessel 352, such as plasma. If desired, the first collection port 56 may be used to remove substances having a medium density such as platelets.

The outlet portion 51 includes a barrier 38 for substantially blocking flow of the intermediate density substances, such as the buffy coat, which as discussed above, consists of white blood cells and platelets. Preferably, the barrier 38 extends completely across the outlet portion 51 in a direction generally parallel to the axis of rotation. The first collection port 56 is positioned immediately upstream of barrier 38, downstream of the inlet portion 48, to collect the intermediate density substances blocked by the barrier 38.

Radially inner and outer edges of the barrier 38 are spaced from radially inner and outer walls of the separation vessel 352 to form a first passage 40 for collection of lower density substances if desired, such as plasma, at a radially inner position in the outlet portion 51 and a second passage 66 for higher density substances, such as red blood cells, at a radially outer position in the outlet portion 51. The second and third collection ports 58 and 60 are positioned downstream of the barrier 38 to collect the respective low and high density substances passing through the first and second passages 40 and 66.

The interface port 61 is also positioned downstream of the barrier 38. During a separation procedure, the interface port 61 removes the least dense of the most dense substances in the outlet portion 51 to thereby control the radial position of the interface between the buffy coat layer 82 and the red blood cell layer 86 and plasma layer 84 in the outlet portion 51.

First port 56 may be used to remove platelets if desired, or may be used to remove the buffy coat layer 82 and a portion of the red blood cell layer next to the buffy coat layer which may contain some contaminating white blood cells. Although the second and third collection ports 58 and 60 and the interface control port 61 are shown downstream of the barrier 38, one or more of these elements may be upstream of the barrier 38. In addition, the order of the collection ports 56, 58, 60, and the interface port 61 along the length of the outlet portion 51 could be changed. Further details concerning the structure and operation of the separation vessel 352 is described in U.S. Pat. No. 6,053,856 to Hlavinka, which has been incorporated herein by reference.

Disposable Set: Extracorporeal Tubing Circuit

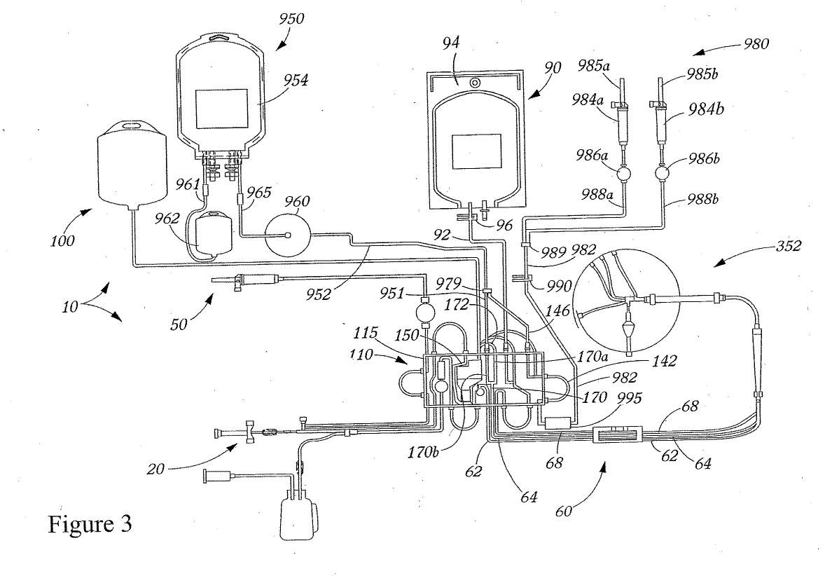

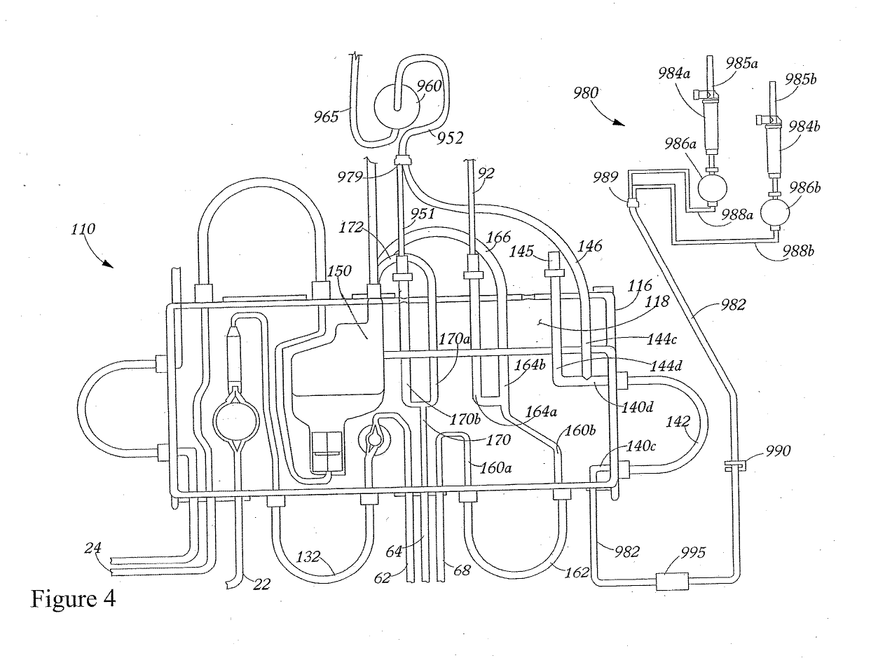

As illustrated in FIGS. 3 and 4, a preconnected extracorporeal tubing circuit 10 is shown which may include a cassette assembly 110 and a number of tubing/collection assemblies 20, 50, 60, 100, 90, 950 and 980 interconnected therewith. Preferably, a blood removal/return tubing assembly 20 provides a single needle interface between a donor 4 (see FIG. 1) and the remainder of the tubing circuit 10 (although a two-needle set-up may also be used, not shown). At least two lines 22, 24 are preferably provided in assembly 20 (see FIG. 4) for removal of blood from and return of components to the donor. This embodiment includes a cassette assembly 110, which is interconnected between the tubing assembly 20 which connects the donor 4 thereto, and blood inlet/blood component tubing line sub-assembly 60 which provides the interface between cassette assembly 110 and blood processing vessel 352. Three lines 62, 64 and 68 are shown in FIGS. 3 and 4 for transport of blood and components to and from the processing vessel 352. An anticoagulant tubing assembly 50, a plasma collection tubing and bag assembly 90, a red blood cell collection assembly 950, a vent bag tubing line sub-assembly 100, and an additive solution assembly 980 are also interconnected with cassette assembly 110 in this embodiment. As will be appreciated, the extracorporeal tubing circuit 10 and blood processing vessel 352 are pre-interconnected to combinatively yield a closed, pre-sterilized disposable assembly for a single use.

The disclosures of the above-listed patents include numerous further details of an apheresis system for use with the present invention. Such details are not repeated here except generally for certain of those which may relate particularly to red blood cell (hereafter, RBC) collection and/or other RBC processes. Other blood component separation and collection processes are discussed at various points herein where they may be involved in or somewhat related to features of the present disclosure.

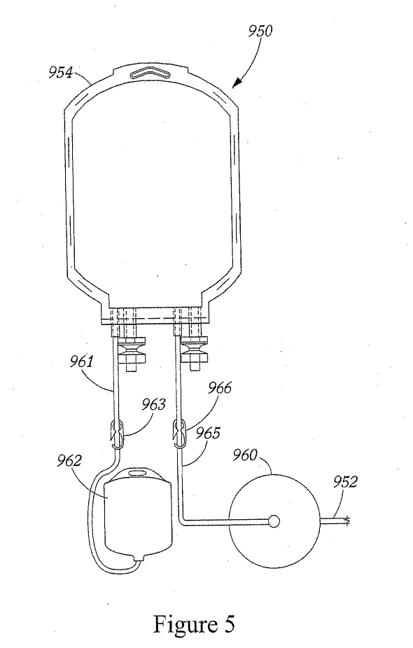

For a particular example, emanating from vessel 352 is an RBC outlet tubing line 64 of the blood inlet/blood component tubing assembly 60 which is interconnected with integral RBC passageway 170 of cassette 115 of cassette assembly 110 (see FIGS. 3 and 4). The integral RBC passageway 170 includes first and second spurs 170a and 170b, respectively. The first spur 170a is interconnected with RBC return tubing loop 172 to return separated RBCs to a donor 4 as well as the buffy coat and the red blood cells located next to the buffy coat which contain at least some white blood cells. For such purpose, the RBC return tubing loop 172 is preferably interconnected to the top of a blood return reservoir 150 of the cassette assembly 110. The second spur 170b may, as preferred herein, be connected with an RBC collection tubing assembly 950 (see FIGS. 3 and 4, for example) for collecting RBCs during use. RBC collection tubing and bag assembly 950 preferably includes RBC collector tubing line 951 which communicates with spur 170b, a second collector tubing line 952 communicating with line 951, an RBC filtration sub-assembly including an RBC leukoreduction filter 960, an RBC collection reservoir or bag 954, and an air removal bag 962. Bag 954 is connected to the filter 960 by tubing line 965. An optional clamp 966 (see FIG. 5) may be included on line 965. The air removal bag 962 is attached to the RBC collection bag 954 by a tubing line 961 which may have an optional clamp 963, (FIG. 5), attached thereto. The RBC collection tubing line, filter and container sub-assembly 950 is a preconnected part of the disposable assembly 10.

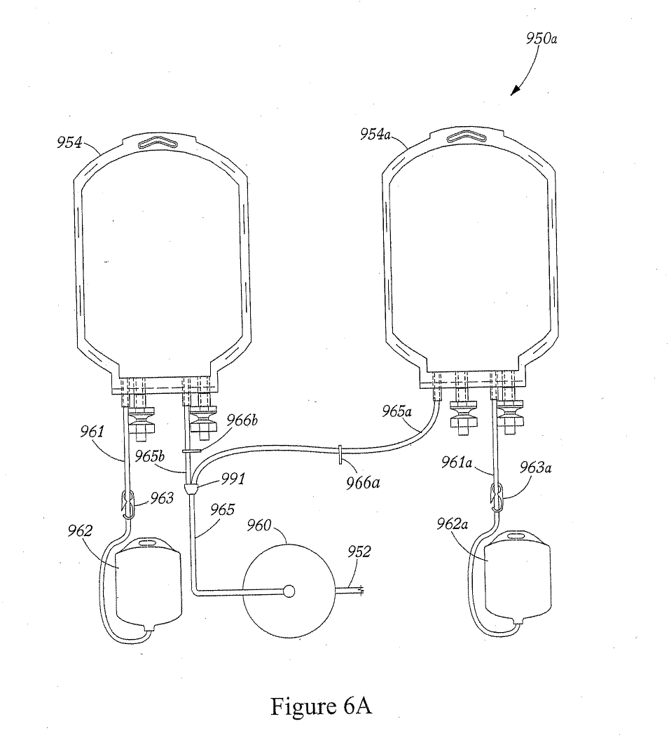

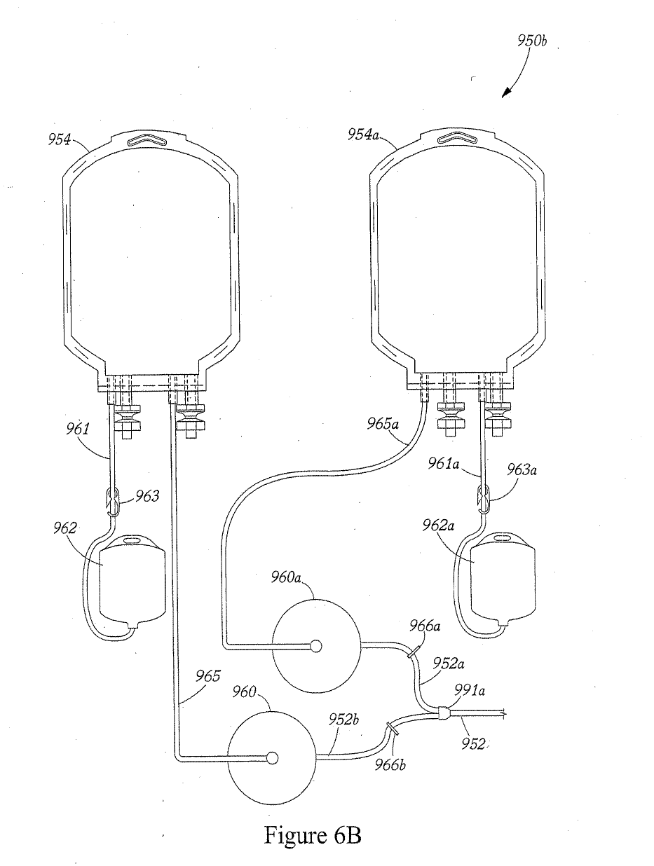

An alternative tubing set filter and collection bag assembly 950a is shown in FIG. 6A and includes a second collection bag 954a connected via a Y-type of connection 991 to filter 960, via the branch tubing line 965a. A further air bag 962a is preferably connected to the second bag 954a via a tubing line 961a. Slide clamps 966a and 966b are used to direct flow to the desired bag. More details particularly as to the use hereof will be set forth below.

A further alternative embodiment is shown in FIG. 6B, which embodiment is an assembly 950b which also includes a second collection bag 954a with associated componentry (e.g., air bag 962a, etc.), and a second filter 960a, in addition to filter 960 described above. Filter 960a is connected via lines 952a and 965a between bag 954a and incoming line 952. A branch or Y connector 991a allows for split flows between branch 952a and branch 952b which leads to the first filter 960. Also slide clamps 966a and 966b may again be used to direct the flow to the respective filters.

The embodiment shown in FIG. 1 includes a connected pair of additive solution bags 970; however the alternative embodiments of FIGS. 3 and 4 preferably have an additive fluid tubing assembly 980 for attachment to and delivery of additive fluid(s) such as sterile saline solution(s), or additive plasma or additive storage solution, for example, to the collected or collecting product in bag system 950 as described in farther detail below. As shown in FIGS. 3 and 4, the additive fluid assembly 980 includes at least an additive fluid inlet tubing line 982 attached to the cassette 110 in fluid communication with an internal additive fluid passageway 140c which is in turn connected to an additive fluid tubing loop 142 which is connected back to the cassette 110 and an internal additive fluid passageway 140d. Two further internal passageways or spurs 144c and 144d and tubing 145 and 146 are also shown in the alternative embodiment of FIGS. 3 and 4. These passageways 140c, 140d and 144c, 144d and tubing loops/tubing 142, 145 and 146 are as shown, preferably similar structurally to the platelet passageways described in various of the above-referenced U.S. Patents, though they may take other forms as well. Indeed, the alternative internal passageway 144d and tubing 145 of the embodiment of FIGS. 3 and 4 may as shown, be blocked off to disallow any fluid flow therein or therethrough. Note, although no outlet tubing line is connected thereto in this embodiment, these flow channels could correspond to a platelet or other blood component collection line as shown in FIG. 2. Though similar structurally in many ways, when referring to the embodiment of FIGS. 3 and 4, the component elements thereof will be referred to as additive fluid elements as opposed to platelet assembly components. This alternative naming convention will also be used for other component elements which could be referred to in connection with either the platelet assembly or the additive fluid assembly; for example, the platelet or additive fluid inlet pump (described in the art) will hereafter be referred to as an additive solution pump. Note, one further distinction is the connection of tubing line 146 to tubing lines 951 and 952 via connector 979.

The additive fluid assembly 980 further preferably includes one or more (as shown) spike assemblies 984a, 984b with respective spikes 985a, 985b and associated sterile barrier devices 986a, 986b and tubing connection lines 988a, 988b which may be connected to tubing line 982 via a Y-connector 989 as shown. Note, it may be that only one of one or more of the above devices may be necessary; e.g., perhaps only one sterile barrier device may be used even with more than one bag of solution. One or more slide clamp(s) 990 and/or a level sensing or fluid detection apparatus 995 may also be included.

The cassette assembly 110 further includes a pump-engaging, additive fluid inlet tubing loop 142 interconnecting the first respective integral additive fluid passageway 140c and a second integral additive fluid passageway 140d. The second integral or additive fluid passageway 140d includes first and second spurs 144c, 144d, respectively. The second spur 144d of the second additive fluid passageway 140d (FIGS. 3 and 4) is interconnected with additive fluid tubing 146 to deliver additive fluid through the RBC outlet line 952 for ultimate delivery to the filter 960 and then to the bag 954. The cassette member 115 also includes an integral frame corner 116 defining a window 118 therethrough. The frame corner 116 includes access openings in window 118 for receiving and orienting the tubing segments including, for example, connector 145 and additive solution tubing 146 in predetermined spaced relationships within window 118 for ultimate engagement with a valve/clamp member on apheresis device 6. Such a valve/clamp will, when activated, control flow through loop 142.

In an intervening portion of the cassette 115, a plasma tubing 68 of blood inlet/blood component tubing assembly 60 (see FIGS. 3 and 4) interconnects with a first integral plasma passageway 160a (see FIG. 4) of cassette assembly 110 (note, this is preferably a plasma collection sub-system; however, other components such as platelets could alternatively be collected here or with a similar arrangement). Cassette assembly 110 further includes a pump-engaging, plasma tubing loop 162 interconnecting the first integral plasma passageway 160a and a second integral plasma passageway 160b. The second integral plasma passageway 160b includes first and second spurs 164a and 164b. The first spur 164a is interconnected to the plasma collection tubing assembly 90 via tubing line 92. The plasma collection tubing assembly 90 may be employed to collect plasma during use and includes plasma collector tubing 92 and plasma collection bag 94. A slide clamp 96 (see FIG. 3) may be provided on plasma collector tubing 92. The second spur 164b of the second integral plasma passageway 160b is interconnected to a plasma return tubing loop 166 to return plasma to donor/patient 4. For such purpose, the plasma return tubing loop 166 is interconnected to the top of the blood return reservoir 150 of the cassette assembly 110. As is understood, one or more types of uncollected blood components, e.g., plasma and/or platelets, collectively referred to as return blood components, will cyclically accumulate in and be removed from reservoir 150 during use. Here also, valve/clamp access is made through the frame 116 within window 118 of cassette assembly 110 to maintain the plasma collector tubing 92 and plasma return tubing loop 166 in a predetermined spaced relationship within window 118 for flow control therethrough.

Most portions of the tubing assemblies 20, 50, 60, 90, 100, 950, 950a, 950b and/or 980 and cassette assembly 110 are preferably made from plastic components including, for example, polyvinyl chloride (PVC) tubing lines, that may permit visual observation and monitoring of blood/blood components therewithin during use. It should be noted that thin-walled PVC tubing may be employed for approved, sterile docking (i.e., the direct connection of two pieces of tubing line) for the RBC collector tubing lines 952 and 965, as may be desired and/or for an RBC storage solution spike assembly 980. In keeping with one aspect of the invention, all tubing lines are preconnected before sterilization of the total disposable assembly to assure that maximum sterility of the system is maintained. Note, a highly desirable advantage to preconnection of all of the elements of the tubing circuit including the filter and collection bag sub-assembly 950 involves the complete pre-assembly and then sterilization hereof after pre-assembly such that no sterile docking is later necessary (spike addition of storage solution excepted). Thus, the costs and risks of sterile docking are eliminated. Alternatively, thicker-walled PVC tubing may be employed for approved, sterile docking RBC collector tubing lines 952 and/or 965, inter alia.

As mentioned, a cassette assembly 110 in the embodiment of FIG. 4, may be mounted upon and operatively interface with the pump/valve/sensor assembly 1000 of a blood component separation device 6 during use. Further details of an apheresis system set-up including the loading and interaction of a disposable assembly 8/10 with a blood component separation device 6, may be found in the above-listed patents, inter alia, and are not exhaustively repeated here.

Operation of Extracorporeal Tubing Circuit and Blood Component Separation Device

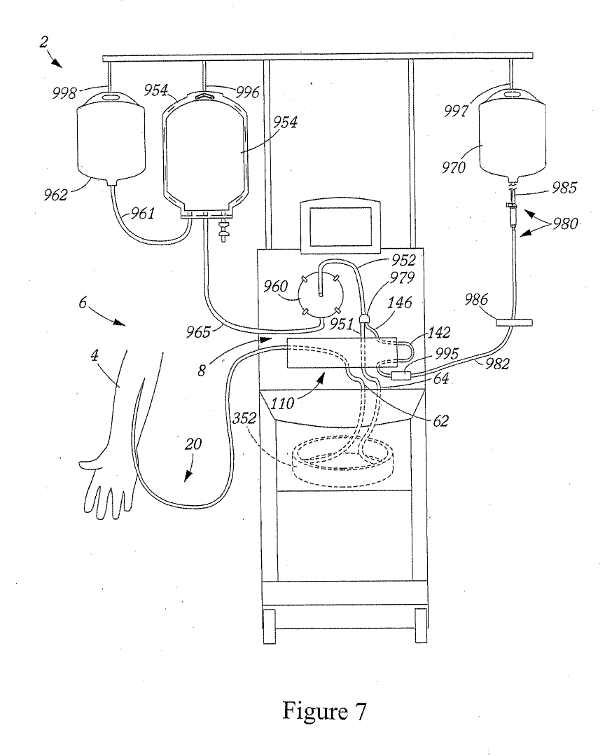

Priming and various other operations of the apheresis process are preferably carried out as set forth in the above-listed patents, inter alia. However, certain basic features are also described generally here with particular reference to the schematic diagrams of FIGS. 7, 8, and 9, as well as with continuing reference to FIGS. 1-6.

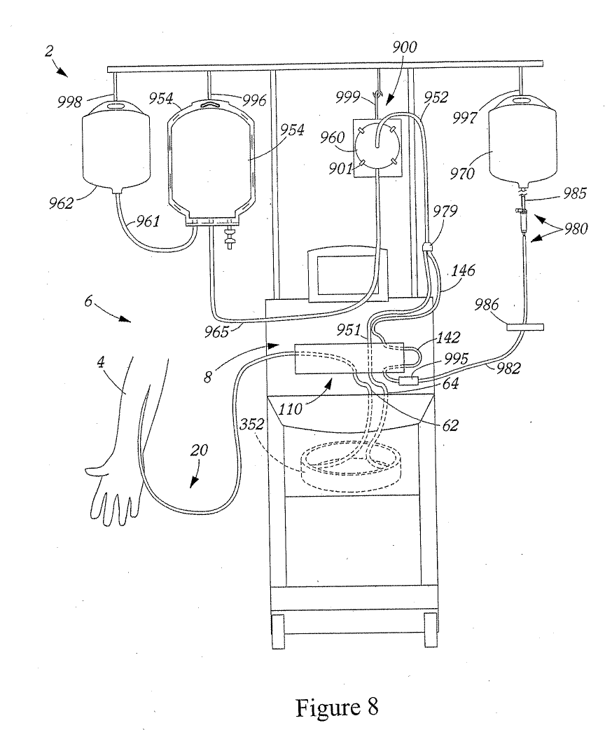

For example, during a blood removal submode, whole blood will be passed from a donor 4 into tubing line 22 of blood removal/return tubing assembly 20 and is then transferred to blood component separation device 6 (see generally FIG. 7). At device 6, the blood is flowed, preferably pumped via loop 132 (see FIG. 4), to the processing vessel 352 (schematically shown in dashed lines in FIG. 7 or in FIG. 2) via the cassette assembly 110 and line 62 of the blood inlet/blood component tubing assembly 60 (FIGS. 3 and 4). Separation processing then occurs preferably on a substantially continuous basis in vessel 352; i.e., blood substantially continuously flows therein, is continuously separated and continuously flows as separated components therefrom. After separation processing in vessel 352 (though separation is continuously occurring), blood components which are not going to be collected are transferred from the processing vessel 352 to and through cassette assembly 110, into and may then accumulate in reservoir 150 (FIGS. 3 and 4) of cassette 110 up to a predetermined level at which the blood component separation device 6, in a single needle operation, may (though in a continuous system, need not) pause the blood removal submode and initiate a blood return submode wherein these uncollected and/or treated components may be returned to the donor 4. As such, these accumulated components may be transferred into the blood return tubing line 24 of blood removal/return tubing assembly 20 and back into the donor 4. During the single needle blood return mode, when the accumulated return blood components in reservoir 150 are removed down to a predetermined level, blood component separation device 6 will then automatically end the blood return submode. This preferably will also automatically serve to reinitiate or continue the blood removal submode. The cycle between blood removal and blood return submodes will then continue until a predetermined amount of RBCs or other collected blood components have been harvested. In an alternative dual needle scheme, as is known in the art, blood may be continually removed from and blood components continually returned to a donor 4. Note, the detailed mechanisms for such operations, including controlling the pumps, for example, are not shown or described in detail herein, particularly not in the schematic views of FIGS. 7 and 8.

Note also that certain components may be collected simultaneously or consecutively one after the other. In one example, platelets and plasma may be collected prior to collection of RBCs. In the primary example shown in FIGS. 1 and 3-4 and 7, 8 and 9, only two components are shown being collected, RBCs in the RBC sub-assembly 950 and plasma (or platelets) in the other collection assembly 90. When a sufficient quantity of one or the other is collected, further separated portions of such a component are preferably returned to the donor with any other uncollected components, until a sufficient quantity of all components are collected. It is further understood that only RBCs can be collected with all other components including plasma being returned to the donor.

With specific reference to FIGS. 3 and 4, in normal operation, whole blood will pass from the donor 4 through the needle and blood removal tubing assembly 20, cassette assembly 110 and blood inlet tubing line 62 to processing vessel 352. The whole blood will then be separated in vessel 352. As shown in FIG. 2, a buffy coat stream containing MNCs (or a stream containing plasma or platelets) may be separated herein and be either collected in a collector assembly (not shown), or diverted to reservoir 150 for ultimate return to the donor. Further, some red blood cells including the red blood cells located at the buffy coat- RBC interface may be separated in and passed, preferably pushed from vessel 352 through RBC outlet tubing line 64, through cassette assembly 110 and, in return mode, into reservoir 150. These RBCs containing contaminating MNCs may be returned to the donor 4, leaving MNC-reduced RBCs in the vessel. In an alternative, during an RBC collection procedure described below, separated MNC-reduced RBCs will be delivered to RBC collector tubing, bag and filter assembly 950 through tubing lines 951 and 952 for collection. The RBC collection protocol may also include a MNC-reduced RBC filtration process using the preconnected leukoreduction filter 960 in line with and prior to RBC collection bag 954. This procedure will be described further below.

Aphersis Protocol

One protocol, which may be followed for performing an apheresis procedure relative to a donor 4 utilizing the described system 2, will now be summarized. Initially, an operator loads the disposable plastic assembly 8 in and/or onto the blood component separation device 6. According hereto, the operator hangs the various bags (e.g., collection bag 954 (and 94, if used); see FIG. 7, described further below) on the respective hooks (see hook 996 of FIG. 7, e.g.) of the blood component separation device 6. If one is used, the operator then also loads the cassette assembly 110 on the machine 6 and/or the blood processing vessel 352 within the channel housing 204 as mounted on the centrifuge rotor assembly 568 in the machine 6.

With the extracorporeal tubing circuit 10 and the blood processing vessel 352 loaded in the described manner, the donor 4 may then be fluidly interconnected with the extracorporeal tubing circuit 10 by inserting an access needle of the needle/tubing assembly 20 into the donor 4 (see, e.g., FIG. 7). In addition, the anticoagulant tubing assembly 50 (see FIG. 3) is primed and the blood removal/return tubing assembly 20 is primed preferably with blood from the donor 4 as described in one or more of the above-listed patents. The blood processing vessel 352 is also primed for the apheresis procedure, preferably also according to processes such as those described in the same above-listed patents. In one embodiment, a blood prime may be used in that blood will be the first liquid introduced into the blood processing vessel 352. During the priming procedure, as well as throughout the remainder of the apheresis procedure, blood may be continuously flowed into the vessel 352, blood component types are preferably continuously being separated from each other and one or more of these is also preferably continuously removed from the blood processing vessel 352, on a blood component type basis. Preferably, at all times during the apheresis procedure, from priming onward, a flow of blood is substantially continuously provided to the blood processing vessel 352 and at least one type of separated component is continuously removed.

It should be noted that when the centrifuge rotor is spinning (as it preferably will be whenever blood is disposed within the blood processing vessel) it will impart centrifugal forces on the blood which will then separate into three primary component layers around the blood processing vessel: a first innermost layer containing at least plasma, a second intermediate layer of “buffy coat” which contains at least platelets and mononuclear cells (MNCs) and a third outermost layer containing primarily red blood cells. It should be noted however, that due to the close sizes of red blood cells and leukocytes, the RBC layer closest to the buffy coat layer interface (at the outermost layer) will contain at least a portion of WBCs. This red blood cell layer partially overlaps with the buffy coat layer.

The buffy coat layer is generally found on the interface between the red blood cell layer and the plasma layer (see element 82 of FIG. 2). Because centrifugal separation will less effectively separate the white blood cells from the red blood cells due to their close size as mentioned above, there is likely to be white blood cell contamination of at least a portion of the the red blood cells closest to the buffy coat layer. As discussed above, this fraction or layer is called the red blood cell layer which partially overlaps with the buffy coat layer (see element 83 of FIG. 2).

Although separation and collection of various components may be performed, RBCs are the component of the most interest in the current invention, and thus the separation and collection protocol will continue with a description of the collection and filtration hereof. It is understood that RBCs may also be the only component collected with all other components being returned to the donor.

In turn, such separated blood components may be selectively collected in corresponding storage reservoirs (not shown) or immediately or after a minor delay returned to the donor 4 during respective blood return submodes (or substantially constantly in a two-needle setup). In this regard, and in one approach where more than one blood component is to be collected, such as plasma and/or platelets, blood apheresis system 2 may be used to collect other components during a time period(s) separate from the collection of red blood cells. These components may also be collected simultaneously. Note, if other components are collected prior to RBCs, then RBCs separated during any such other component phase may be diverted back to the donor and not filtered. Preferably, only collected MNC-reduced RBCs will be filtered in the current embodiment (though therapeutic filtration for a particular donor/patient may also be performed). By removing the other component layers, especially the buffy coat layer and the red blood cell layer which partially overlaps with the buffy coat layer, the remaining MNC-reduced red blood cells will be less contaminated with lymphocytes, and will be able to be filtered more efficiently to remove any remaining white blood cells. The buffy coat layer and the RBC layer containing the at least a portion of MNCs can either be returned to the donor, or collected into a storage reservoir or collection bag and further processed.

In any event, the RBC collection procedure is preferably controlled via control signals provided by blood collection device 6. Such an RBC collection procedure may include a setup phase and a collection phase. During such a setup phase, the blood apheresis system 2 may be adjusted automatically to establish a predetermined hematocrit in those portions of the blood processing vessel 352 and extracorporeal tubing circuit 10 through which separated RBCs will pass for collection during the RBC collection phase. A desirable resulting hematocrit for RBC collection may be between about 70 and about 90 or even up to 95+, and may be established at about 80. The term high hematocrit refers to those separated, undiluted RBCs leaving the separation vessel 352. Dilution with storage solution to a different (generally lower) collected hematocrit may follow.

Additionally, blood component device 6 may, during the set-up phase, divert the flow of separated RBCs flowing through RBC tubing line 64 through return tubing loop 172 and into blood return reservoir 150 for return to the donor 4 until the desired hematocrit is established in the separation vessel 352.

Also during the set up phase, the blood component separation device may divert the flow of the buffy coat layer 82 and the portion of the red blood cells 83 which are closest to the buffy coat layer either back to the donor or into a collection bag for further processing. By removing this portion of the red blood cells and the buffy coat layer from the blood processing vessel, the majority of the mononuclear cells will be removed. The red blood cells remaining in the separation vessel 52 are known as mononuclear cell reduced red blood cells.

The increased efficiency of removing the buffy coat layer and the layer of RBCs next to the buffy coat layer is shown in the table below.

| | Contaminating cell count after | Contaminating cell |
|---|---|---|
| | leukoreduction without removal of | count after leukoreduction |
| | buffy coat layer and the RBCs next | of MNC-reduced |
| N (=22) | to buffy coat layer | RBCs |
| 1 | 4.9 | 0.31 |
| 2 | 2.4 | 0.02 |
| 3 | 7.8 | 0.04 |
| 4 | 0.3 | 0.22 |
| 5 | 0.2 | 0.05 |
| 6 | 5.9 | 0.02 |
| 7 | 3.2 | 0.05 |
| 8 | 0.1 | 0.09 |
| 9 | 3.0 | 0.06 |
| 10 | 4.7 | 0.01 |
| 11 | 2.0 | 0.04 |
| 12 | 11.3 | 1.22 |
| 13 | 1.1 | .022 |
| 14 | 15.3 | 4.03 |
| 15 | 1.3 | 0.37 |
| 36 | 0.4 | 0.07 |
| 17 | 14.7 | 0.44 |
| 18 | 0.5 | 0.03 |
| 19 | 0.3 | 0.06 |
| 20 | 2.6 | 0.16 |
| 21 | 3.1 | 2.37 |
| 22 | 2.3 | 0.04 |

As can be seen in the table above, the step of removing the buffy coat layer and the RBCs located next to the buffy coat layer produced a final RBC product with much lower WBC contamination as compared to the final RBC product produced without the removal step.

In order to establish the desired packing factor and/or hematocrit for the separated MNC-reduced RBCs, the operating speed of centrifuge rotor assembly 568 (see FIG. 1) may be selectively shed via control signals from blood component separation device 6, and the blood inlet flow rate to vessel 352 may be selectively controlled by blood component separation device 6 controlling the speeds of the respective pump assemblies (not shown or described in detail here). More particularly, increasing the rpms of centrifuge rotor assembly 568 and/or decreasing the inlet flow rate will tend to increase the packing factor and/or hematocrit, while decreasing the rpms and/or increasing the flow rate will tend to decrease the packing factor and/or hematocrit. As can be appreciated, the blood inlet flow rate to vessel 352 may effectively be limited by the desired packing factor or hematocrit.

To establish a desired anticoagulant (AC) ratio, blood component separation device 6 provides appropriate control signals to the anticoagulant pump so as to introduce anticoagulant into the blood inlet flow at a predetermined rate. Relatedly, it should be noted that the inlet flow rate of anticoagulated blood to blood processing vessel 352 may be limited by a predetermined, maximum acceptable anticoagulant infusion rate (ACIR) to the donor 4. As will be appreciated by those skilled in the art, the predetermined ACIR may be established on a donor-specific basis (e.g. to account for the particular total blood volume of the donor 4). To establish the desired total uncollected plasma flow rate out of blood processing vessel 352, blood collection device 6 provides appropriate control signals to the plasma (and platelet) pump assembly(ies), This may also serve to increase the hematocrit in the separated RBCs.

In one embodiment, the desired high hematocrit for the separated RBCs will be between about or approximately 75 and about 85 and will preferably be about or approximately 80; although, again higher hematocrits may be available as well. Then, where a centrifuge rotor assembly 568 may present a defined rotor diameter of about 10 inches, and where a blood processing vessel 352 is utilized, as described hereinabove, it has been determined that in one preferred embodiment channel housing 204 can be typically driven at a rotational velocity of about 3000 rpms to achieve the desired RBC hematocrit during the setup and red blood cell collection phases. Correspondingly, the blood inlet flow rate provided by pumping through loop 132 to vessel 352 may preferably be established at below about 65 ml/min. The desired hematocrit can be reliably stabilized by passing about two whole blood volumes of vessel 352 through vessel 352 before the RBC collection phase is initiated.

To initiate the MNC-reduced RBC collection phase, blood component separation device 6 provides an appropriate control signal to the RBC divert valve assembly (not shown) so as to direct the continuous outflow of the separated MNC-reduced high hematocrit RBCs removed from blood processing vessel 352 via line 64 into the RBC collection system 950 through tubing lines 951 and 952, and filter 960 into collection container 954 via line 965.

As may be appreciated, the MNC-reduced, separated RBCs are not pumped out of vessel 352 for collection, but instead are flowed out vessel 352 and through extracorporeal tubing circuit 10 by the pressure of the blood inlet flow to vessel 352. The inlet blood is pumped into vessel 352 via loop 132 of cassette 110. The separated MNC-reduced RBCs are pushed or pressed out of the vessel 352.

During the RBC collection phase, the inlet flow into vessel 352 will likely be limited by the above-noted maximum acceptable ACIR to the donor 4. The desired inlet flow rate may also be limited by that necessary to maintain the desired packing factor and/or hematocrit, as also discussed. In this regard, it will be appreciated that relative to the setup phase, the inlet flow rate may be adjusted slightly upwards during the RBC collection phase since not all anticoagulant is being returned to the donor 4. That is, a small portion of the AC may remain with the small amount of plasma that is collected with the high hematocrit RBCs in RBC reservoir 954.

According to the present invention, the relatively high hematocrit (high-crit) MNC-reduced RBCs optionally may be diluted and then filtered as soon as the blood is separated or very soon after having been separated within vessel 352. Alternatively, the MNC-reduced RBCs may be filtered without dilution in a high-crit state. The phrase high-crit refers to the state of the separated MNC-reduced RBCs as they emerge from the separation vessel 352. In the substantially continuous centrifugal separation process as described here, a freshly separated stream of MNC-reduced RBCs is substantially continually flowing out of the vessel 352, first through tubing line 64, to and through cassette assembly 110 and then through lines 951 and 952 (where they optionally may be joined by diluting storage solution) to the filter 960 and then through line 965 to bag 954 (see FIG. 7). Preferably, these freshly separated MNC-reduced RBCs will be continuously flowing from vessel 352 through filter 960 and then into collection bag 954 (or also into bag 954a, see FIGS. 6A and 6B). Thus, in the described embodiment, white cell/leukocyte filtration will have begun and is continued simultaneously with or during the overall continuous separation process, prior to collection. More description of this will be set forth in further detail below.

Note, the phrase freshly-separated is intended to describe the newly-separated blood components in and as they emerge from the mechanical separation system such as device 6 and separation vessel 352. It also includes the state of these same separated components for a reasonable length of time after removal from the mechanical separation device such as from vessel 352. Thus, for example, a reasonable length of time may include the entire apheresis procedure which may last up to (and perhaps exceed) two (2) or more hours during which filtration may be substantially continuously performed. Two further terms used herein have similar distinctions, namely, “recently removed” and “soon after.” Recently removed is referred to herein primarily relative to that blood taken from the donor which may be immediately taken and processed in a mechanical separation system, or which may have been taken and held subject to a reasonable non-long-term-storage type of delay prior to separation processing in a device such as device 6. Similarly, “soon after” is used in like manners relative to both of these circumstances as well, as, for example, when separated blood components may be removed from the separation vessel, e.g. soon after separation (whether in continuous or batch mode).

In any event, from the standpoint of the donor 4 and machine 6, following the separation, filtration and collection processes of the desired quantity of red blood cells, blood separation device 6 may then provide a control signal to the RBC divert assembly so as to divert any further RBC flow back to the donor 4 via loop 172, reservoir 150 and return line 24. Additionally, if further blood processing, by apheresis centrifugation here, is not desired, rinseback procedures may be completed. Additionally, once the minimum desired RBCs have been diverted into filtration/collection assembly 950 and after filtration completion, the red blood cell collection reservoir 954 (and/or the entire sub-assembly 950) may then be disconnected from the extracorporeal tubing circuit 10. Filter 960 may also be removed herewith or separately or remain attached and disposed of with the cassette 110 and other remaining bags or tubes. However, according to the present invention, a storage solution will be, perhaps during and/or after filtration of the RBCs, added to the RBC flow in tubing line 952 to the filter 960 ultimately to the red blood cell reservoir or bag 954. Preferably, a spike connection to one or more storage solution bag(s) 970 (see FIGS. 1 and 7) through a spike 985 is used. This process will also be described further below. Such storage solutions or additive solutions may advantageously facilitate storage of the RBCs for up to about forty-two days at a temperature of about 1-6° C. In this regard, acceptable storage solutions include a storage solution generically referred to in the United States as Additive Solution 3 (AS-3), available from Medsep Corp. located in Covina, Calif.; and/or a storage solution generically referred to in Europe as SAG-M, available from MacoPharma located in Tourcoing, France. It is also possible to use saline before, after or during the filtering process described below which, prior to storage, could be replaced with the desired storage solution. Alternatively saline could be used to flow through the filter 960 to the cassette assembly 110.

The storage additive solution may be and preferably is contained in a discrete storage solution bag 970 that can be pre-connected, or is separate and may selectively be later interconnected to the tubing circuit 10 via line 982, preferably through a spike connection 985. In an alternative embodiment, such selective interconnection may be provided via sterile-docking to tubing line 982 as an example (process not shown) utilizing a sterile connecting device (not shown). By way of example, one such sterile connecting device to interconnect a tubing line 982 to such a storage solution container 970, is that offered under the trade name “TSCD” or “SCD™ 312” by Terumo Medical Corporation located in Somerset, N.J. In the alternative above, the selective interconnection may be established utilizing a sterile barrier filter/spike assembly 980. The use of such a sterile barrier filter/spike assembly 980 facilitates the maintenance of a closed system, thereby effectively avoiding bacterial contamination. By way of example, the mechanical, sterile barrier filter 986 (FIG. 7) or 986a or 986b in such an assembly 980 may include a porous membrane having 0.2 micron pores. Pumping via a tubing loop 142 may then provide for selectively flowing solution through tubing line 982 and connecting tubing line 146 for introduction of the storage solution into the RBC line 952 and filter system 950.

In order to ensure the maintenance of RBC quality, the collection RBC bag 954, and the storage solution and the anticoagulant used during blood processing should be compatible. For example, the collection RBC reservoir 954 may be a standard PVC DEHP reservoir (i.e. polyvinyl chloride-diethylhexylphthallate) such as those offered by the Medsep Corporation. Alternatively, other PVC reservoirs may be employed. Such a reservoir may utilize a plasticizer offered under the trade name “CITRIFLEX-B6” by Moreflex located in Commerce, Calif. Further, the anticoagulant utilized in connection with the above-described red blood cell collection procedures may be an acid citrate dextrose-formula A (ACD-A).

Nevertheless, according to an embodiment of the present invention as introduced above, the storage solution may be flowed after and/or added to the flow of separated MNC-reduced red blood cells flowing in lines 951 and 952, and flow therewith to and through the filter 960. Filter 960 will remove the majority of the remainder of white blood cells which are left in the MNC-reduced red blood cells. More particularly leukoreduction filtering is desired to establish a white blood cell count of <5×10⁶ white blood cells/unit (e.g. about 250 ml.) to reduce any likelihood of febrile non-hemolytic transfusion reactions. Moreover, such filtering will more desirably achieve a white blood cell count of <1×10⁶ white blood cells/unit to reduce any risk of HLA (i.e. human leukocyte A) sensitization and/or other serious side reactions. Studies have also shown positive effects for pre-storage leukocyte reduction in improving the functional quality of erythrocytes during storage and in decreasing the occurrence of alloimmunization in patients receiving multiple transfusions, as well as being favorable in metabolism reactions such as intra-erythrocyte ATP and/or extracellular potassium levels declining more slowly in filtered products. Perhaps more important is the reduction of transfusion transmitted disease, especially cytomegalovirus (CMV) and/or HIV, inter alia.

Accordingly, the red blood cell collection container 954 receives, in one embodiment, RBCs and additive solution from the red cell filter 960 such that high hematocrit (preferably Hct between 70 and 90 and/or approximately equal to 80), freshly separated MNC-reduced red blood cells alone or together with additive solution are preferably pushed through filter 960 and into the ultimate RBC collection bag 954. Such pushed filtration is shown in FIGS. 7, 8 and 9, as will be described further below. The red cell filter 960 and collection bag sub-assembly 950 is preferably preconnected to the tubing circuit 8 as part of the disposable assembly 10 (to avoid the costs and risks of sterile docking) as shown in FIGS. 1, 3 and 4 in accordance with the teachings of this invention. The red blood cell filter 960 may also be added to the previously existing disposable systems to form a post-manufacturing-connectable disposable assembly using special new kits or commercially available filter/bag kits such as those available under the trade name “Sepacell” from Asahi Corp and/or Baxter, Inc. and/or “RC100”, “RC50” and “BPF4”, etc., from Pall Corp., located in Glencove, N.Y., inter alia. In either event, the red cell filter/bag sub-assembly is preferably connected (pre- or post-) to the tubing circuit 8 through a tubing line 951 and/or 952 as shown.

Referring now primarily to FIGS. 3, 4 and 5, the procedure for the filtration of MNC-reduced RBCs freshly separated and collected from the apheresis process is as follows. These freshly separated MNC-reduced RBCs are either in an undiluted, high-hematocrit state (Hct approximately 80) during the preferred filtration process, followed by additive solution or storage solution, or are filtered in a mixed state with additive solution added to the RBC flow in line 952 at the connection 979. Moreover, storage solution may be flowed through the filter 960 prior to any MNC-reduced RBCs (this may enhance the filtration efficacy) and, as noted above, may optionally be flowed through the filter after leukoreduction of the RBCs to be added to the collected RBCs in bag 954. In an embodiment, no matter when the additive or storage solution initially flows through the filter, it is preferable to run a sufficient amount of solution through the filter 960 after MNC-reduced RBC filtration to attempt to displace any RBCs remaining in the volume of the filter 960 for collection.

Either simultaneously with the substantially continuous separation and collection process (i.e., as soon as high hematocrit (high-crit) MNC-reduced RBCs are separated from other components and pushed out of vessel 352 to cassette 110 and not diverted back to the donor), or soon after a desired minimum quantity of other blood components have been collected, if desired, the RBC collection/filtration system 950 is activated to filter the MNC-reduced RBCs. This collection process is activated by switching the clamp/valve of device 6 to stop diversion flow through loop 172 and allow flow through line 951 to line 952 and filter 960.

In either case; simultaneously with the continuous collection in bag 954 from the separation vessel 352, or soon after completion of any other non-RBC collection process(es), the MNC-reduced RBCs are flowed preferably by intrinsic pressure pushing (non-active pumping) through filter 960. As such, collection bag 954 may be hung at a level above both the separation vessel 352 and the filter 960 (see FIGS. 5-8) so that the continuously flowing MNC-reduced RBCs are allowed to move upwardly from vessel 352 through the filter 960 and into the collection bag 954. One embodiment of this is shown in FIG. 7, where the collection bag 954 is hung from a hook 996 of the machine 6 in known fashion. Tubing line 965 depends downwardly therefrom and is shown as connected to the filter 960, out of the top of which extends the next tubing line 952 which ultimately connects downwardly to the cassette 110 via line 951.

Any air from bag 954, or air caught between the incoming filtered RBCs and bag 954 is ultimately removed to air removal bag 962 through tubing line connection 961. The air is evacuated to air removal bag 962 prior to the flow of the incoming RBCs or is evacuated by the flow of the incoming RBCs. It is also understood that air can also be vented prior to even the separation process by initially running the return pump, (not shown) of the apheresis system. It is also understood that removal of air may also be achieved by other known methods, including, for example, hydrophobic vents and/or by-pass lines. It is desirable to perform the filtering of the MNC-reduced RBCs according to the present invention directly on the machine 6 during the apheresis separation process and without pre-cooling or pre-storing the RBCs. In such a case, these procedures are thus performed without the previously conventional steps of intermediate separation/collection and cooling and storing overnight at 4° C.

Then, either after completion of or during and/or even before the filtration in either of these embodiments, namely, the simultaneous collection and filtering, or in the filtering and collection soon after any other component collection processes, storage solution is flowed to and through the filter and/or added to the MNC-reduced RBCs. Again, this may be performed either before and/or during and/or after completion of the filtration of the otherwise high hematocrit MNC-reduced RBCs through filter 960, although it is preferred that an amount of additional additive or storage solution displace the volume of the filter to recover any residual RBCs therefrom. In particular, a storage solution bag 970 has been connected (by pre-connection or by spike or sterile welding) as depicted in FIGS. 1, 7, 8 and 9, the clamp 990 is opened (if any such optional flow-stopping member is used; see FIGS. 3 and 4) to allow the introduction of the storage solution into tubing line 982 and pumped via tubing loop 142 through additive solution tubing 146 and into tubing line 952 via connector 979. The storage solution thus will be pumped from bag 970 through filter 960 and into collection bag 954. If pumped during collection, the solution may be metered into and mix with and dilute the high-crit MNC-reduced RBCs in line 952 prior to filtration. The rate of mixing can be controlled by pumping via loop 142. However, the storage solution may be pumped through the filter 960 also before and/or after all of the undiluted MNC-reduced RBCs have been filtered therethrough to assist in the filtration and/or to chase the MNC-reduced RBCs and move any MNC-reduced RBCs caught in the filter out of the filter to the collection bag 954. Such a storage solution chase may be used also after the metering of storage solution into a pre-filtration MNC-reduced RBC flow (as described above) as well. Again, all of the steps in operating the MNC-reduced RBC filtration system 950 may be performed during the overall apheresis component separation procedure and thus need not be subjected to a cooled, time-delayed environment.

It should be noted that storage solution does not need to be pumped through the filter. Storage solution may also be flushed through the filter manually, using gravity.

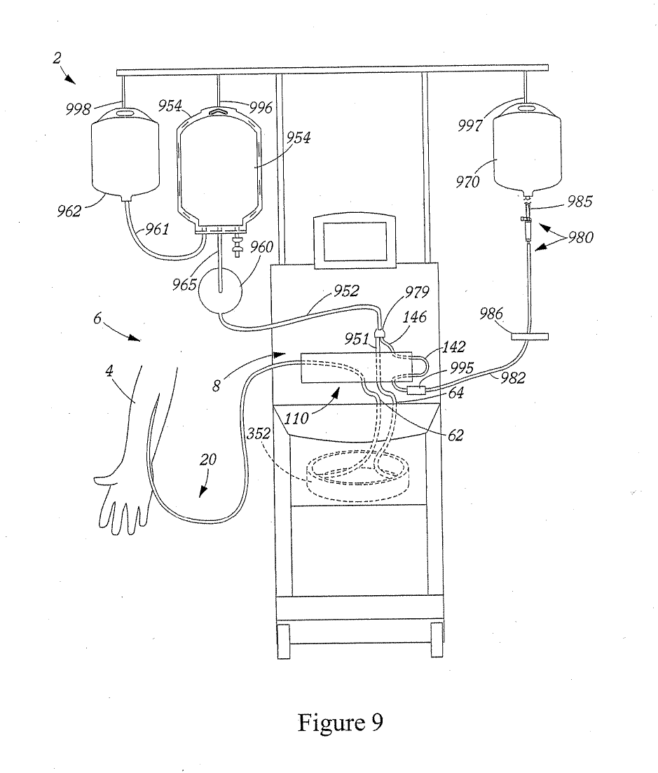

One embodiment of the storage solution addition step is shown in FIGS. 7, 8 and 9. Note, other component collection processes are not shown here (i.e., whether simultaneous or consecutive collection processes for other components (e.g., plasma and/or platelets) are used is not depicted or described here). In FIGS. 7, 8 and 9, the collection bag 954 is shown attached to the upper hook 996 and the air bag 962 hung on another hook 998 (note, air bag 962 may not need to be hung from a hook but could have air bled thereto after the other steps in the process as suggested below). Then, a storage solution bag 970 can be hung from yet another hook 997 so that when connected and hung as shown in FIGS. 7 and 8, storage solution can flow down through tubing line 982 and through sterile barrier 986 through pump loop 142, connecting lines 146 and 952 and then through filter 960 and ultimately into collection bag 954. Although flow of both storage solution and MNC-reduced RBCs is shown entering the filter 960 in the downward direction in FIGS. 7 and 8 and the upward direction in FIG. 9, it is also understood that flow to the filter 960 can be in any direction desired, including, but not limited to sideways. This flow against gravity is possible because the MNC-reduced RBCs are pushed through the filter.

Alternatively, the embodiment shown in FIG. 8 also includes a depiction of the placement of the filter 960 in a substantially fixed position on device 6. In this embodiment flow will remain in a downward direction to aid in priming the filter 960. Clips or other restraining devices 901 are shown holding filter 960 in place. The further steps of having collected or simultaneously collecting components other than the RBCs in bag 954 and/or the alternatives of simultaneously pumping solution into the flow of MNC-reduced RBCs and/or having completed filtration thereof through filter 960 prior to the addition of storage solution to filter 960 and bag 954 are not easily separately shown in the Figures; however, flow control over the storage solution will preferably be made by a pump on device 6 engaging loop 142.

The embodiment shown in FIG. 9 depicts the filter 960 hanging from bag 954 without attachment to device 8. This embodiment allows flow of both storage solution and MNC-reduced RBCs in the upward direction to and through the filter 960. Although not shown in FIG. 9, a bracket or clip or other restraining device like that shown as element 901 in FIG. 8 may be used to surround filter 960 to provide mechanical support and to insure the filter is placed in the correct orientation.

In either event, upon completion of filtration and/or chasing with additive solution, the collection bag 954 may be separated from the rest of the set 8. Optional clamp 966 may be closed prior to such a separation. The separation may be made by RF sealing the tubing line 965 above the filter 960 or line 952 below the filter 960 and then separating in accordance with U.S. Pat. Nos. 5,345,070 and 5,520,218, inter alia, along the RF-sealed portion of the tubing line. Other well known methods can also be used to close the tubing line and then also separate the RBC collection system 950 from the remainder of the disposable assembly 8. An RBC collection system 950 which would be remaining after one such severing, e.g., below the filter 960, is shown schematically in FIGS. 5 and/or 6A or 6B (see below).

With respect to FIG. 5 it is noted that tubing line 965 may be a segmented tubing line that is further sealed to provide sample segments as is well known. It is also understood that tubing line 961 in addition to tubing line 965 or alternatively to tubing line 965 may also be segmented to again provide the desired samples for blood tying and other optional purposes.

Several advantages can be realized utilizing the preconnected disposable assembly and the above-described procedure for high-crit MNC-reduced red blood cell collection and filtration. Such advantages include: consistency in final RBC product volume and hematocrit; reduced exposure of a recipient if multiple units of blood products are collected from a single donor and transfused to a single recipient; reduced time requirements for RBC collection and filtration, including collection of double units of red blood cells if desired, and reduced risks of leukocyte contamination of the final RBC product due to the filter becoming clogged with MNCs which get pushed through the filter into the previously filtered RBCs, thus causing recontamination of the previously filtered RBCs. Further advantages include a system which is less complicated and requires less human interaction. Less human interaction is advantageous because it decreases the possibilities of human contamination.

In order to assist an operator in performing the various steps of the protocol being used in an apheresis procedure with the apheresis system 2, the apheresis system 2 preferably includes a computer graphical interface 660 as illustrated generally in FIG. 1. The graphical interface 660 may preferably include a computer display 664 which has “touch screen” capabilities; however, other appropriate input devices (e.g., keyboard) may also be utilized alone or in combination with the touch screen. The graphics interface 660 may provide a number of advantages, but may preferably, at least, assist the operator by providing pictorials of how and/or when the operator may accomplish at least certain steps of the apheresis and/or filtration procedures.

For example, the display screen optionally may sequentially display a number of pictorials to the operator to convey the steps which should be completed to accomplish the filtering procedure described here. More particularly, a pictorial image optionally may be shown on the screen to pictorially convey to the operator when and/or how to hang the respective RBC and solution bags 954 and/or 970 on the machine 6, initially and/or during use with a storage solution dilution and/or flush (see FIGS. 7 and 8, for example). One or more pictorials may also be provided to instruct the operator when to open or close clamps to begin the filtration process, and/or to visually ensure that the filtration process has appropriately begun simultaneously or during RBC collection. One or more pictorials may also be used to instruct the operator when to connect the spike assembly 980 to a storage solution container 970 and/or when to open a clamp or break a frangible connector (if included) after and/or during the MNC-reduced RBCs flow through filter 960, to thus initiate the flow of the storage solution through the filter 960 and flush any residual MNC-reduced RBCs therethrough. One or more pictorials may also be used to instruct the operator when the tube line 965 leading to the RBC collection bag 954 should be sealed such that the RBC collect bag 954, and the remaining elements of RBC storage assembly 950 may be separated and/or removed from the disposable assembly 10 and/or from the device 6. A similar pictorial can instruct when to seal the air tube 961 to isolate the RBC collection bag 954 from the air bag 962 and the rest of the system after the filtration and flushing and air handling procedures may be completed.

Note, a further advantage of the presently described system includes the manner of handling air. More specifically, the present invention eliminates the prior need for the vents and/or by-pass methods and/or apparatuses of conventional red blood cell filters. Moreover, the present invention is capable of delivering this advantage with no reduction in and/or perhaps an increase in the recovery of RBCs that historically have been trapped inside the filtration device.

A means used by the present invention to deliver this advantage is through the provision of a storage solution flush through the filter 960 after the MNC-reduced RBCs have finished filtering therethrough. The storage solution may then be able to wash MNC-reduced RBCs caught therein out of the filter and then into the collection bag 954. Prior devices relied upon vents or by-pass mechanisms to assist in pushing out any RBCs disposed in the filter. Note, though not preferred or needed, vents or by-passes could still be used with the current pushed filtration process, and also with and/or in lieu of the storage solution flush after filtration. Thus such vents or by-passes may be optional features to the described system if it is desired to purge the filter 960 with air or with a combination of air and fluid.

In any event, elimination of the need for vents or by-passes also reduces other prior difficulties such as inadvertent allowances of excess air into the system. Extra air in the present system will not stop or slow the flow of blood or storage solution through the filter in the present invention. The extra air will then be caught within the collection bag 954 and may thus be removed at the end of the overall process to the air bag 962 (air moved thereto by bag positioning or squeezing, etc.). Then, also, because neither vents nor by-passes are required in this embodiment, failures with respect to the operation of such vents are not of concern since the subsequent storage solution flush recovers the RBCs from the filter without the previously desired use of a vent or by-pass. Consequently, also, the filter may be disposed at any of a plurality of alternative vertical dispositions above or below the vessel 352 and/or the collection bag 954. Operation of the present invention should not be hindered by such alternative placements. It is understood, however, that air could also be used to chase either the RBCs or additive solution through filter 960 as described above.

Although the instant invention eliminates the need for by-passes it is understood that one could be provided in the extracorporeal tubing circuit to by-pass the filter 960 in the event the leukoreduction is terminated or is not desired. Similarly it is understood that an optional pressure relief valve or vent could be added to prevent pressure build up in parts of the system including the filter.

The volume of storage solution to be used may, however, be modified depending upon the relative lengths of tubing lines used and/or the air that gets into the system. For example, if 100 ml of storage solution is desired to be mixed with the end product RBCs in collection bag 954 then some certain volume more than 100 ml of storage solution would preferably be fed into the system to compensate for the tubing lengths and the volume of the filter. The amount of solution may be chosen such that 100 ml would go into the collection bag 954 with the additional amount remaining in the tubing line and filter between the cassette 110 and the collection bag 954.

Note, a storage solution dilution during RBC filtration and/or flush after filtration completion are the primary alternatives taught here. However it is possible that storage solution flow into bag 954 may be begun at other times as well as, for example, prior to starting the high-crit or diluted MNC-reduced RBC pushed filtration. Pulsed and/or intermittent flows may also be desirable to assist in removing final volumes of RBCs from the filter 960.

Another alternative introduced hereinabove involves the use of other extracorporeal blood processing systems. Although the preference is for a continuous flow apheresis system, as described here, which includes returning some components back to the donor, batch flow and non-return systems are also useable herewith. For example, a batch mode processor takes in a certain quantity of whole blood which was previously collected from a donor at some point before the separation process is begun. The batch mode processor separates the blood into components (in a centrifuge bowl, e.g.) and then passes the separated components to collection containers. The separated components may also be given back to the donor. The filtration process of the present invention could foreseeably nevertheless operate in substantially the same manner such that the separated MNC-reduced RBCs would nonetheless exist in a substantially high hematocrit state as they are flowed from the separation mechanism, at which point these high-crit separated MNC-reduced RBCs could be flowed to a junction with a storage solution tubing line and from there be passed directly or soon thereafter to and through a filter 960 to be collected ultimately in a collection bag 954. Though continuity may be reduced (or substantially removed), the principles of firstly removing the buffy coat layer and the RBCs located next to the buffy coat layer before pushed filtration (high-crit or diluted) during or soon after the overall separation and collection remain the same. Note, even if flow through the filter 960 stops at any point, or a plurality of points, this does not appear problematic here where any air entry therein is handled by ultimate capture in the air bag 962.

Smaller scale separation and collection devices are also envisioned to be useful herewith. For example, various separation devices (whether centrifugal or membrane or other types) are designed to separate only RBCs and plasma (with the remainder usually remaining in the RBC product), and these can take on smaller scale mechanizations. Nevertheless, the present invention is useful herewith as well in that MNC-reduced RBCs separated hereby may also be freshly push-filtered at high and/or diluted hematocrits. The principle of push-filtering such MNC-reduced RBCs during or soon after the overall separation and collection process remains the same here as well. Thus, whether continuous or in batch mode, a flow of high-crit or diluted, freshly-separated MNC-reduced RBCs can be push-flowed from the separation device immediately or soon after previous processing therein, to and through filter 960 to a collection bag 954.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A method for providing red blood cells from whole blood using an apheresis system comprising the steps of:
separating the whole blood into blood component layers within a separation vessel wherein the blood component layers comprise at least a plasma layer, a buffy coat layer and a red blood cell layer;
removing the plasma layer from the separation vessel;
removing the buffy coat layer from the separation vessel; and
removing a portion of the red blood cell layer closest to the buffy coat layer to provide a mononuclear cell-reduced red blood cell layer in the separation vessel.

2. The method of claim 1 wherein the whole blood is recently removed from a donor.

3. The method of claim 1 wherein the separating step comprises separating in a continuous manner.

4. The method of claim 1 wherein the separating step comprises separating in a batch wise manner.

5. The method of claim 1 further comprising removing the mononuclear cell-reduced red blood cell layer from the separation vessel.

6. The method of claim 1 further comprising leukoreducing the mononuclear cell-reduced red blood cell layer to create leukoreduced red blood cells.

7. The method of claim 6 wherein the leukoreduced red blood cells are at a high hematocrit.

8. The method of claim 5 wherein the step of removing the mononuclear cell-reduced red blood cell layer further comprises removing the mononuclear cell-reduced red blood cell layer from the separation vessel to a preconnected red blood cell collection bag.

9. The method of claim 8 wherein the step of removing the mononuclear cell-reduced red blood cells from the separation vessel to a preconnected red blood cell collection bag occurs after the mononuclear cell-reduced red blood cells have been leukoreduced.

10. The method of claim 6 wherein the step of leukoreducing the mononuclear cell-reduced red blood cells further comprises pushing the mononuclear cell-reduced red blood cells through a leukoreduction filter preconnected to the separation vessel.

11. The method of claim 10 wherein the step of pushing the mononuclear cell-reduced red blood cells through the leukoreduction filter further comprises pushing by non-gravity forces.

12. The method of claim 10 wherein the step of pushing the mononuclear cell-reduced red blood cells through the leukoreduction filter further comprises pushing by indirect pumping forces.

13. The method of claim 2 wherein the step of removing the buffy coat layer further comprises returning the buffy coat layer to the donor.

14. The method of claim 1 wherein the step of removing the buffy coat layer further comprises collecting the buffy coat layer in a preconnected collection container for further processing.

15. The method of claim 2 wherein the step of removing a portion of the red blood cell layer closest to the buffy coat layer further comprises returning the portion of the red blood cell layer closest to the buffy coat layer to the donor.

16. The method of claim 1 wherein the step of removing a portion of the red blood cell layer closest to the buffy coat layer further comprises collecting the portion of the red blood cell layer closest to the buffy coat layer in a preconnected collection container for further processing.

17. The method of claim 10 further comprising flowing a solution through the preconnected leukoreduction filter after the pushing step to displace high hematocrit mononuclear cell-reduced red blood cells from the filter.

18. The method of claim 17 wherein the flowing step comprises flowing a storage solution.

19. The method of claim 6 wherein the separating step comprises separating in a continuous manner and wherein the step of leukoreducing the mononuclear-reduced red blood cells occurs at least partially during the step of separating in a continuous manner.

20. A method for increasing the performance of a leukoreduction filter for leukoreducing cells collected during an apheresis procedure comprising the steps of:
separating in a blood separation vessel whole blood into
an innermost layer containing at least plasma;
an intermediate layer containing at least buffy coat; and
an outermost layer containing at least red blood cells;
removing the innermost layer from the blood separation vessel;
removing the intermediate layer from the blood separation vessel; and
removing a portion of the outermost layer closest to the intermediate layer to create a mononuclear cell-reduced red blood cell layer in the blood separation vessel to be further leukoreduced using the leukoreduction filter.

21. The method of claim 20 wherein the step of removing a portion of the outermost layer closest to the intermediate layer increases the performance of the leukoreduction filter by removing mononuclear cells which may clog the leukoreduction filter.

22. The method of claim 20 wherein the step of removing a portion of the outermost layer closest to the intermediate layer increases the performance of the leukoreduction filter by removing mononuclear cells which may be too numerous to be effectively removed by the leukoreduction filter.
